(19) United States
(12) Patent Application Publication
(10) Pub. No.: US 2013/0203808 A1
(43) Pub. Date: Aug. 8, 2013

(54) Drug for degradation and treatment of biofilms

(71) Applicant: Mary C. Kline, Glen Rock, PA (US)

(72) Inventor: Mary C. Kline, Glen Rock, PA (US)

(21) Appl. No.: 13/757,734

(22) Filed: Feb. 1, 2013

Related U.S. Application Data
(60) Provisional application No. 61/594,210, filed on Feb. 2, 2012.

(51) Int. Cl. A61K 31/47; A61K 45/06; A61K 31/366

(52) U.S. Cl. CPC A61K 31/47; A61K 31/366; A61K 45/06
USPC 514/312

(57) ABSTRACT

The invention comprising decoquinate and one or both of a redox drug or macrolide drug for prophylaxis or treatment of certain biofilms and plaques in humans and animals.

### CROSS REFERENCE TO RELATED APPLICATIONS

Provisional application 61/594,210

### BACKGROUND OF THE INVENTION

1. Field of the Invention

The invention relates to the treatment and degradation of polymicrobial biofilms and plaques in humans and animals, and reduction of planktonic state biofilm-forming pathogens.

2. Description of Related Art

Biofilms exist in and on all humans and animals. Beneficial and pathogenic microbes secrete extracellular polymeric substances which form a matrix around groups of individual cells. Naturally occurring biofilms have multiple genera within the matrix; they are polymicrobial. Pathogens have multiple forms such as aerobic, anaerobic, and persister cells in the biofilm. Single cell pathogens can join the biofilm, or leave the biofilm. Groups of pathogens in matrix can detach from the biofilm. Pathogens within the biofilm can reproduce. Biofilms are complex and changing entities within the body and on its surface.

Single-celled pathogens within a biofilm communicate by way of signal molecules. Known as quorum sensing, these signals are both intra- and inter-specie. Signal molecules can alter biofilm functions such as attachment to the substratum, producing matrix to enclose more pathogens, and causing cells to leave the biofilm returning to planktonic state. Signal molecules are thought to turn on virulence of pathogens.

Diffusion of antimicrobials into the biofilm microbes is slowed by the matrix and layers of aerobic microbes by up to 1000 times the rate available in planktonic state. Degradation of the outer layers of matrix is essential to drug interaction with the anaerobic microbes in the center of the matrix.

Recent study of Protomyxzoa (FL1953) indicate a possible relationship to several diseases where biofilms have formed in the vasculature.

### BRIEF SUMMARY OF THE INVENTION

The invention comprising the heretofore veterinary anti-protozoal decoquinate and one or both of a redox drug or macrolide drug for prophylaxis or treatment of certain biofilms and plaques in humans and animals. Decoquinate or its metabolites reduce biofilms which sequester active and persister cells of various pathogens making the pathogens more susceptible to the redox and/or macrolide drugs. Decoquinate and its metabolites have not been identified in the literature as related or similar to Pseudomonas Quinilone Signals, PQS. The drug combination or its metabolites act to disrupt normal biofilm quorum sensing, decreasing the structure of the biofilm or plaque.

### DETAILED DESCRIPTION OF THE INVENTION

Decoquinate or its metabolites function similar to Pseudomonas Quinolone Signals, PQS, disrupting normal biofilm quorum sensing, and decreasing the structure of certain biofilms and plaques.

Decoquinate cidal effects on protozoal aerobic pathogens on the outer layers of matrix create cavities for different classes of drugs to interact with anaerobic microbes. Creating cavities and holes in the matrix decrease the cell to cell signaling, weaken the biofilm, and accelerate diffusion of drugs to additional microbes. Killing select microbes within the biofilm allows macrophages access to additional attachment sites and genera.

Biofilms spread to new areas by both releasing individual cells, and by detaching multiple cells in matrix. Decoquinate is cidal to some stages of individual protozoal cells released to planktonic state, and degradation of detached ‘blubs’ of matrix will slow the spread of pathogenic biofilms. Prophylactic treatment of biofilms may be beneficial for certain medical conditions, particularly vascular biofilm and plaque accumulations.

Apicomplexan protozoa such as Babesia, Plasmodium, and Protomyxzoa are known to produce or inhabit biofilms. Decoquinate is cidal to some life stages and forms of Apicomplexan protozoa. Combining decoquinate with redox or macrolide antimicrobials will enhance the cidal affects on multiple species.

The invention is for oral administration of the drug in various forms such as tablet, gelatin capsule, capsule, suspensions, paste or lozenges. The invention is also for intravenous administration, absorptive patch, and topical administration for biofilms in skin wounds by spray, ointment, or inclusion in bandages. Decoquinate has a very high therapeutic index in domestic animals. The dosage for humans is weight dependant, generally between 0.1 mg/kg body weight and 20 mg/kg body weight per day. The invention includes the combination of this drug with other classes of drug such as redox and/or macrolide drugs to further degrade and treat Apicomplexan protozoa in biofilms and in planktonic state.

The invention comprising decoquinate: 3-Quinolinecarboxylic acid,6-(decyloxy)-7-ethoxy-4-hydroxy-,ethyl ester; Ethyl 6-(decyloxy)-7-ethoxy-4-hydroxy-3-quinolinecarboxylate [18507-89-6]; and its metabolites with one or both of a redox drug and a macrolide drug, or pharmaceutically acceptable salts thereof. Pharmaceutical excipients may be added as necessary to obtain the dosage or form.

The redox drug is such as metronidazole: 2-(2-methyl-5-nitroimidazol-1-yl)ethanol; or artemisinin or a derivative of artemisinin or pharmaceutically acceptable salts thereof.

The macrolide drug is such as azithromycin: 2R,3S,4R,5R,8R,10R,11R,12S,13S,14R)-13-[(2,6-dideoxy-3-C-methyl-3-O-methyl-a-L-ribo-hexopyranosyl)oxy]-2-ethyl-3,4,10-trihydroxy-3,5,6,8,10,12,14-heptamethyl-11-[[3,4,6-trideoxy-3-(dimethylamino)-b-D-xylo-hexopyranosyl]oxy]-1-oxa-6- azacyclopentadecan-15-one; or clarithromycin: (3R,4S,5S,6R,7R,9R,11R,12R,13S,14R)-6-[(2S,3 R,4S,6R)-4-(dimethylamino)-3-hydroxy-6-methyloxan-2-yl]oxy-14-ethyl-12,13-dihydroxy-4-[(2R,4R,5S,6S)-5-hydroxy-4-methoxy-4,6-dimethyloxan-2-yl]oxy-7-methoxy-3,5,7,9,11,13-hexamethyl-oxacyclotetradecane-2,10-dione or pharmaceutically acceptable salts thereof.

1. A drug for treating, decreasing or degrading polymicrobial biofilms and plaques in humans comprising decoquinate: ethyl 6-decoxy-7-ethoxy-4-oxo-1H-quinoline-3-carboxylate [18507-89-6]; and a redox antimicrobial drug; and optionally a macrolide antimicrobial drug; or pharmaceutically acceptable salts thereof; with pharmaceutical excipients necessary for administration.

2. The metabolites of the drug of claim 1 for treating, decreasing, or degrading polymicrobial biofilms and plaques in humans.

3. The drug of claim 1 for treating, decreasing, or degrading polymicrobial biofilms and plaques in animals.

4. The metabolites of drug of claim 1 for treating , decreasing, or degrading polymicrobial biofilms and plaques in animals.

5. The drug of claim 1 for treating, decreasing or degrading polymicrobial biofilms on surface wounds of humans or animals by direct application or contained in bandages.

6. The drug of claim 1 for treating or reducing planktonic state biofilm-forming pathogen infections.

7. The drug of claim 1 administered as a method for cell to cell signaling to disrupt biofilm functions.

8. The drug of claim 1 wherein the redox antimicrobial drug is artemisinin or a derivative of artemisinin.

9. The drug of claim 1 for prophylactic treatment of biofilms and plaques in humans and animals.
